(19) United States
(12) Patent Application Publication
(10) Pub. No.: US 2025/0069715 A1
(43) Pub. Date: Feb. 27, 2025

(54) SYSTEMS AND METHODS FOR AUTOMATIC GENERATION OF THERAPY NOTES AND PERFORMANCE

(71) Applicant: Neuromersive, Inc., Fort Worth, TX (US)

(72) Inventors: Veena Somareddy, Euless, TX (US); Salome Jesus-Jose Marquez, Eulesss, TX (US)

(73) Assignee: Neuromersive, Inc., Fort Worth, TX (US)

(21) Appl. No.: 18/453,494

(22) Filed: Aug. 22, 2023

(51) Int. Cl. G16H 15/00; G16H 10/60; G16H 40/67

(52) U.S. Cl. CPC G16H 15/00; G16H 10/60; G16H 40/67

(57) ABSTRACT

Systems and techniques for automatically generating therapy notes based on objective sensor data and/or subjective data using artificial intelligence (AI) models. In embodiments, therapy session data, including objective sensor data collected by one or more sensors during a therapy session and subjective therapy session data associated with subjective impressions from a therapist related to the therapy session of the therapy patient, is obtained. Therapy notes are generated based on the therapy session data, which may include applying the therapy session data to a language model to generate the therapy notes. The therapy notes may be output to at least one entity, including details based on the objective sensor data and the subjective therapy session.

### TECHNICAL FIELD

The present invention relates generally to generative data systems, and more particularly to a system for automatically generating therapy notes and tracking performance of therapy sessions.

### BACKGROUND

Health related issues, unfortunately, are a part of life and touch almost every person, whether directly or indirectly. Healthcare has advanced tremendously over the years, thanks to new discoveries and technological advances. We now are able to cure many diseases and provide recovery where before might not have been possible. One area that has advanced a lot over the years is the area of therapeutic care. Therapy can help a patient's recovery by providing a controlled environment where a patient can perform exercises (e.g., physical or mental) under the supervision of a therapist that may allow the patient to recover from an illness or injury. The exercises performed by the patient may be designed or known to promote the recovery of the patient and may vary from patient to patient, depending on the specific characteristics of the patient, the injury or illness, the therapy goal, the patient's limitations, environmental limitations, etc.

Typically, therapy may involve more than one therapy session. In each therapy session, a patient may be put through one or more exercises designed to promote the recovery of the patient. The number of therapy sessions, and/or the specific exercises or goals that may be performed during each session, may be set in a therapy plan designed by the therapist and/or a doctor depending on the overall goal of the therapy. As therapy is extremely patient-specific (e.g., because every patient is different and may respond differently to the therapy), ensuring that the therapy sessions are well-documented is of extreme importance, and such importance cannot be understated.

For example, whether a patient is making progress or whether a particular exercise is being effective are conclusions that can only be obtained by ensuring that the therapy sessions are documented well, as missing information may make such determinations impossible. Moreover, therapists need to demonstrate not only patient progress, but also the details of the therapy sessions, to the patient's medical insurance in order to be paid.

The product of documenting a therapy session is typically referred as therapy notes. However, currently, the process for writing or generating therapy notes is far less than ideal. For example, currently, documenting a session may involve the therapist writing the therapy notes. Typically, and understandably, the therapist may not write the therapy notes for a therapy session until after clinic hours, which may be a long time after the therapy session. This is because therapists are in-demand and their schedule, which is typically packed, oftentimes does not allow them to document a therapy session immediately after the actual therapy session. In fact, some surveys have shown that approximately 90% of therapist write therapy notes after clinic hours, rather than after therapy sessions. Some 35% actually noted that they write the notes even less often than daily.

Even more challenging is writing therapy notes for documenting a therapy session during the actual therapy session, which can be said to be impossible for the therapist, as the therapist is focused on the patient and simply does not have sufficient time for writing therapy notes. Of course, being able to write therapy notes near-real time would be ideal for a therapist and patients, and the benefit of such ability would be fantastic and quite measurable, as things that would otherwise be forgotten by the therapist after clinic hours would find themselves in the therapy notes and could serve to evaluate, update, and/or optimize the therapy plan. As it stands with the current therapy notes generation process, this is not the case.

Furthermore, since therapy notes are typically generated by the therapist, and from the therapist point of view (POV), a therapy session's details as documented in the therapy notes currently are subjective details, rather than objective details. In this, the details in the therapy notes reflect the therapist impressions of the therapy session, such as impressions on how well the therapy session went, how well the patient is progressing, or how well the patient might feel. As such, a therapist inability to capture all details of the therapy session and the subjective nature of the therapy session limits the accuracy, effectiveness, and in some ways usefulness of the therapy notes.

Some solutions have been proposed to alleviate the issues with the current processes for generating therapy notes, but these solutions are not ideal. For example, some proposed solutions may receive a transcript generated by a therapist and generate therapy notes based on the contents of the transcript. Although these solutions allow generation of therapy notes in a particular desirable format, theses solutions still require the therapist to provide a transcript and may still be based on the subjective impressions of the therapist, which does not alleviate the deficiencies of the current therapy generation processes.

### BRIEF SUMMARY

The present disclosure achieves technical advantages as systems and methods that provide functionality for automatically generating therapy notes and tracking performance of therapy sessions based on objective sensor data and/or subjective data using artificial intelligence (AI) models. The present disclosure provides for a system integrated into a practical application with meaningful limitations as an integrated and centralized management system with functionality that provides an enhanced and improved scheme for integrating objective sensor data and subjective data and may leverage ML and/or AI models to automatically generate therapy notes for therapy sessions. In embodiments, objective sensor data collected by one or more sensors during a therapy session, and subjective therapy session data associated with subjective impressions from a therapist related to the therapy session of the therapy patient may be obtained. In embodiments, therapy notes may be generated based on the objective sensor data and the subjective therapy session data, which may include applying the objective sensor data and the subjective therapy session data to a language model (e.g., an AI language model) to generate the therapy notes. The therapy notes may be output to at least one entity, including details based on the objective sensor data and the subjective therapy session. The advantageous result is that the therapy notes may not only be generated automatically, thereby completely or partially relieving the therapist from the task, but the therapy notes may be based also on objective sensor data, not only the subjective impressions of the therapist. In this manner, the subjective impressions of the therapist may be corrected, enhanced, and/or validated using the objective sensor data.

Thus, it will be appreciated that the technological solutions provided herein, and missing from conventional systems, are more than a mere application of a manual process to a computerized environment, but rather include functionality to implement a technical process to replace or supplement current solutions or non-existing solutions for generating therapy notes. In doing so, the present disclosure goes well beyond a mere application the manual process to a computer. For example, currently solutions that merely use a therapist-generated transcript to generate therapy notes, such as using an AI model, may be improved or enhanced by eliminating the need to use the therapist-generated transcript to generate the therapy notes, or at least may improve the current systems by providing a mechanism to correct, enhance, and/or validate the subjective impressions of the therapist, thereby improving the efficiency and/or accuracy of using these systems. Accordingly, the claims herein necessarily provide a technological solution that overcomes a technological problem.

It is an object of the disclosure to provide a method of automatically generating therapy notes for a therapy session of a therapy patient. It is a further object of the disclosure to provide a system for automatically generating therapy notes for a therapy session of a therapy patient, and a computer-based tool for automatically generating therapy notes for a therapy session of a therapy patient. These and other objects are provided by the present disclosure, including at least the following embodiments.

In one particular embodiment, a method of includes automatically generating therapy notes for a therapy session of a therapy patient is provided. The method includes obtaining objective sensor data collected by one or more sensors during the therapy session, obtaining subjective therapy session data associated with subjective impressions from a therapist related to the therapy session of the therapy patient, and generating the therapy notes based on the objective sensor data and the subjective therapy session data. In embodiments, generating the therapy notes may include applying the objective sensor data and the subjective therapy session data to a language model to generate the therapy notes. The method also includes outputting the therapy notes to at least one entity. In embodiments, the therapy notes may include details based on the objective sensor data and the subjective therapy session.

In another embodiment, a system for includes automatically generating therapy notes for a therapy session of a therapy patient is provided. The system comprises at least one processor and a memory operably coupled to the at least one processor and storing processor-readable code that, when executed by the at least one processor, is configured to perform operations. The operations include obtaining objective sensor data collected by one or more sensors during the therapy session, obtaining subjective therapy session data associated with subjective impressions from a therapist related to the therapy session of the therapy patient, and generating the therapy notes based on the objective sensor data and the subjective therapy session data. In embodiments, generating the therapy notes may include applying the objective sensor data and the subjective therapy session data to a language model to generate the therapy notes. The operations also include outputting the therapy notes to at least one entity. In embodiments, the therapy notes may include details based on the objective sensor data and the subjective therapy session.

In yet another embodiment, a computer-based tool for includes automatically generating therapy notes for a therapy session of a therapy patient is provided. The computer-based tool including non-transitory computer readable media having stored thereon computer code which, when executed by a processor, causes a computing device to perform operations. The operations include obtaining objective sensor data collected by one or more sensors during the therapy session, obtaining subjective therapy session data associated with subjective impressions from a therapist related to the therapy session of the therapy patient, and generating the therapy notes based on the objective sensor data and the subjective therapy session data. In embodiments, generating the therapy notes may include applying the objective sensor data and the subjective therapy session data to a language model to generate the therapy notes. The operations also include outputting the therapy notes to at least one entity. In embodiments, the therapy notes may include details based on the objective sensor data and the subjective therapy session.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 is a block diagram of an exemplary system configured with capabilities and functionality for automatically generating therapy notes and tracking performance of therapy sessions based on objective sensor data and/or subjective data using artificial intelligence (AI) models in accordance with embodiments of the present disclosure.

FIG. 2 shows an example of a set of objective sensor data associated with a particular virtual reality game implemented in accordance with embodiments of the present disclosure.

FIG. 3 shows an example a graphical user interface (GUI) configured with capabilities and functionality for presenting therapy notes in accordance with embodiments of the present disclosure.

FIG. 4 shows a high-level flow diagram of operations of a system for providing functionality for automatically generating therapy notes in accordance with embodiments of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood. of course. that this disclosure is not limited to the particular embodiments illustrated herein.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features described herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, as evidenced by issuance of this patent, the prior art fails to disclose or teach the elements and limitations presented in the claims as enabled by the specification and drawings, such that the presented claims are patentable under the applicable laws and rules of this jurisdiction.

Various embodiments of the present disclosure are directed to systems and techniques that provide functionality for automatically generating therapy notes and tracking performance of therapy sessions based on objective sensor data and/or subjective data using artificial intelligence (AI) models. In embodiments, the functionality provided by the features described herein may represent an integrated and centralized management system with functionality that provides an enhanced and improved scheme for integrating objective sensor data and subjective data and may leverage ML and/or AI models to automatically generate therapy notes for therapy sessions. In embodiments, objective sensor data collected by one or more sensors during a therapy session, and subjective therapy session data associated with subjective impressions from a therapist related to the therapy session of the therapy patient may be obtained. In embodiments, therapy notes may be generated based on the objective sensor data and the subjective therapy session data, which may include applying the objective sensor data and the subjective therapy session data to a language model (e.g., an AI language model) to generate the therapy notes. The therapy notes may be output to at least one entity, including details based on the objective sensor data and the subjective therapy session. The advantageous result is that the therapy notes may not only be generated automatically, thereby completely or partially relieving the therapist from the task, but the therapy notes may be based also on objective sensor data, not only the subjective impressions of the therapist. In this manner, the subjective impressions of the therapist may be corrected, enhanced, and/or validated using the objective sensor data.

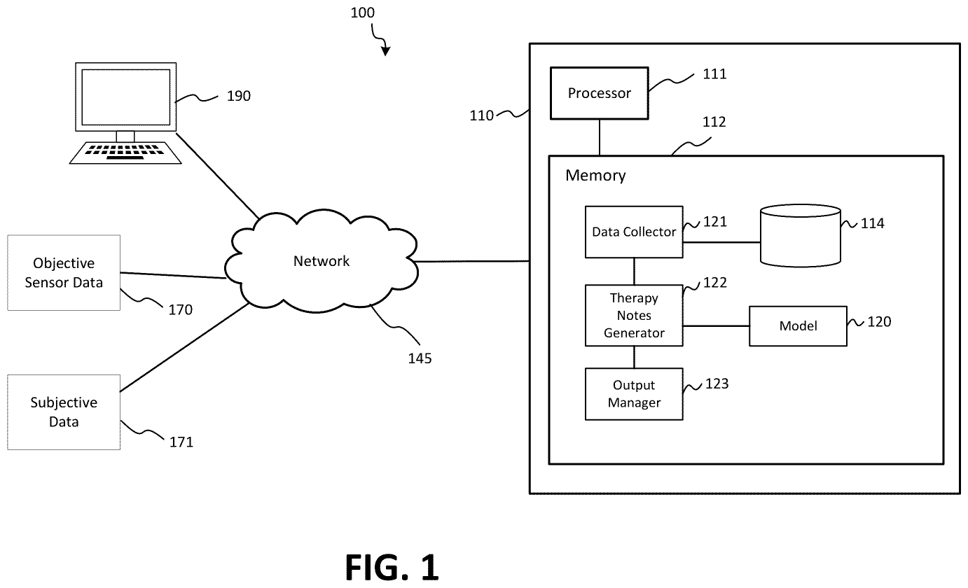

FIG. 1 is a block diagram of an exemplary system 100 configured with capabilities and functionality for automatically generating therapy notes and tracking performance of therapy sessions based on objective sensor data and/or subjective data using AI models in accordance with embodiments of the present disclosure. As shown in FIG. 1, system 100 may include server 110, objective sensor data 170, subjective data 171, at least one user terminal 170, and network 145. These components, and their individual components, may cooperatively operate to provide functionality in accordance with the discussion herein.

It is noted that the functional blocks, and components thereof, of system 100 of embodiments of the present invention may be implemented using processors, electronics devices, hardware devices, electronics components, logical circuits, memories, software codes, firmware codes, etc., or any combination thereof. For example, one or more functional blocks, or some portion thereof, may be implemented as discrete gate or transistor logic, discrete hardware components, or combinations thereof configured to provide logic for performing the functions described herein. Additionally, or alternatively, when implemented in software, one or more of the functional blocks, or some portion thereof, may comprise code segments operable upon a processor to provide logic for performing the functions described herein.

It is also noted that various components of system 100 are illustrated as single and separate components. However, it will be appreciated that each of the various illustrated components may be implemented as a single component (e.g., a single application, server module, etc.), may be functional components of a single component, or the functionality of these various components may be distributed over multiple devices/components. In such embodiments, the functionality of each respective component may be aggregated from the functionality of multiple modules residing in a single, or in multiple devices.

It is further noted that functionalities described with reference to each of the different functional blocks of system 100 described herein is provided for purposes of illustration, rather than by way of limitation and that functionalities described as being provided by different functional blocks may be combined into a single component or may be provided via computing resources disposed in a cloud-based environment accessible over a network, such as one of network 145.

Server 110 may be configured to, in operation according to embodiments, obtain (e.g., receive, extract, etc.) therapy session data (e.g., which may include objective sensor data 170 and/or subjective data 171) (e.g., via network 145 and/or from database 114). The various components of server 110 may cooperatively operate to collect the therapy session data and to apply the therapy session data to a language model (e.g., model 120) to generate the therapy notes. The therapy notes may be output to at least one entity, including details based on the objective sensor data and the subjective therapy session. This functionality of server 110 may be provided by the cooperative operation of various components of server 110, as will be described in more detail below. Although FIG. 1 shows a single server 110, it will be appreciated that server 110 and its individual functional blocks may be implemented as a single device or may be distributed over multiple devices having their own processing resources, whose aggregate functionality may be configured to perform operations in accordance with the present disclosure. Furthermore, those of skill in the art would recognize that although FIG. 1 illustrates components of server 110 as single blocks, the implementation of the components and of server 110 is not limited to a single component and as described above, may be distributed over several devices or components.

It is noted that the various components of server 110 are illustrated as single and separate components in FIG. 1. However, it will be appreciated that each of the various components of server 110 may be a single component (e.g., a single application, server module, etc.), may be functional components of a same component, or the functionality may be distributed over multiple devices/components. In such embodiments, the functionality of each respective component may be aggregated from the functionality of multiple modules residing in a single, or in multiple devices.

As shown in FIG. 1, server 110 includes processor 111 and memory 112. Processor 111 may comprise a processor, a microprocessor, a controller, a microcontroller, a plurality of microprocessors, an application-specific integrated circuit (ASIC), an application-specific standard product (ASSP), or any combination thereof, and may be configured to execute instructions to perform operations in accordance with the disclosure herein. In some embodiments, as noted above, implementations of processor 111 may comprise code segments (e.g., software, firmware, and/or hardware logic) executable in hardware, such as a processor, to perform the tasks and functions described herein. In yet other embodiments, processor 111 may be implemented as a combination of hardware and software. Processor 111 may be communicatively coupled to memory 112.

As shown in FIG. 1, memory 112 includes model 120, data collector 121, therapy notes generator 122, output manager 123, and database 114. Memory 112 may comprise one or more semiconductor memory devices, read only memory (ROM) devices, random access memory (RAM) devices, one or more hard disk drives (HDDs), flash memory devices, solid state drives (SSDs), erasable ROM (EROM), compact disk ROM (CD-ROM), optical disks, other devices configured to store data in a persistent or non-persistent state, network memory, cloud memory, local memory, or a combination of different memory devices. Memory 112 may comprise a processor readable medium configured to store one or more instruction sets (e.g., software, firmware, etc.) which, when executed by a processor (e.g., one or more processors of processor 111), perform tasks and functions as described herein.

In embodiments, memory 112 may be configured to facilitate storage operations. For example, memory 112 may include database 114 configured to store objective sensor data (e.g., objective sensor data collected for a current therapy session and/or objective sensor data collected for previous therapy sessions), subjective data (e.g., subjective data collected for a current therapy session and/or subjective data collected for previous therapy sessions), language models to be used for generating therapy notes (e.g., AI model 120), therapy notes generated for previous therapy sessions, patient-related information, system configuration information, etc. In some embodiments, database 114 may be integrated into memory 112, or may be provided as a separate module. In yet other embodiments, database 114 may be a single database, or may be a distributed database implemented over a plurality of database modules. Database 114 may be configured to store information for a plurality of patients. In some cases, the information may be used in training and learning operations.

Data collector 121 may be configured to collect therapy session data related to a therapy session for which therapy notes are to be generated during operation of system 100. In embodiments, data collector 121 may be configured to collect, receive, extract, or otherwise obtain the therapy session data from one or more sources. For example, in some embodiments, the therapy session data may be obtained by data collector 121 from database 114. In these embodiments, the therapy session data may be previously stored in database 114, and data collector 121 from database 114 may obtain the previously stored therapy session associated with the therapy session from database 114. For example, therapy session data may be generated for a current therapy session and may be stored in database 114. Data collector 121 may access database 114 and may obtain the therapy session data generated for the current therapy session, and/or therapy session data for previous therapy sessions but that may be relevant to the current therapy session. In these embodiments then, the therapy session data may be stored in database 114 prior to being used to generate the therapy notes for the therapy session, in which case the therapy session data may be stored and then retrieved from the storage to be used to generate the therapy notes for the therapy session in accordance with functionality described herein.

In some embodiments, the therapy session data may be used to generate the therapy notes for the therapy session in real-time (or near-real time), in which case the therapy session data may not be stored prior to being used to generate the therapy notes for the therapy session in accordance with functionality described herein.

In embodiments, the therapy session data (e.g., the therapy session data) that may be used to generate the therapy notes for a therapy session may include historical therapy session data. For example, the therapy session data may include therapy session data collected during previous therapy sessions of the therapy patient. In embodiments, data collector 121 may obtain the therapy session data collected during previous therapy sessions of the therapy patient from an electronics healthcare records (EHR) system that may be external to system 100, and/or from database 114.

In embodiments, the therapy session data (e.g., the therapy session data) that may be used to generate the therapy notes may include objective sensor data 170. Objective sensor data 170 may include objective data that is not subjective or based on a subjective impression, but rather, may represent hard data, such as data measured, collected, generated, or obtained from one or more sensors associated with the therapy session for which therapy notes are to be generated during operation of system 100.

In some embodiments, objective sensor data 170 may include data generated from analysis of the data measured, collected, generated, or obtained from one or more sensors associated with one or more therapy sessions of the therapy patient. For example, in embodiments, data collector 121 may be configured to apply one or more analysis models to the data generated, measured, collected, or obtained from one or more sensors associated with the one or more therapy sessions, and to generate, based on the one or more analysis models, at least a portion of objective sensor data 170. In these embodiments, objective sensor data 170 may include, in addition or in the alternative to the actual measured data, an analytical interpretation of the measured data, but based on an objective analytical model.

In some embodiments, objective sensor data 170 may include objective data that is related to subjective data that is observed by the therapist (e.g., objective data related to a particular aspect, where the therapist also observes the aspects and subjective data is generated for the same particular aspect). For example, objective sensor data 170 may include a data point indicating an amount of extension that the therapy patient is able to achieve. This objective data point may represent an extension level measurement. The therapists may observe the same extension, but may instead characterize the extension in terms of “high” or “low.” In this case, the same aspect may be associated with an objective data measurement and a subjective impression. In some embodiments, objective sensor data 170 may include objective data that is not, or cannot be, obtained from observations by the therapists, such as data that cannot be perceived by a human.

In particular embodiments, objective sensor data 170 may include objective therapy session data such as length of session, time in between sessions, length of time between session exercises (e.g., a therapy session may include a plurality of exercises and the length of time between session exercises may indicate a time elapsed between various session exercises), etc. Objective sensor data 170 may include patient data such as age, weight, height, and/or any other characteristic of the therapy patient.

In embodiments, the one or more sensors associated with the therapy session for which therapy notes are to be generated during operation of system 100, and from which objective sensor data 170 may be generated, may include one or more sensors associate with a virtual reality (VR) therapy session. For example, in embodiments, the therapy session for which therapy notes are to be generated during operation of system 100 may include a therapy session in which a VR headset may be used. In embodiments, the VR headset may be used to immerse the therapy patient in a virtual environment, which may be navigated by the therapy patient. During the VR therapy patient, the therapy patient may be required to perform virtual tasks within the virtual environment. The virtual tasks may correspond to physical actions. Thus, by performing the virtual tasks, the therapy patient is encouraged, forced, or otherwise encouraged, to perform the corresponding physical actions, which may be related to a physical exercise. In some embodiments, the virtual tasks may be part of a VR game. In this manner, the VR environment may facilitate the therapy exercises by providing a more entertaining and enjoyable environment for performing actions that result in the patient performing physical exercises. As a consequence, VR therapy is very effective and is growing in popularity. In these embodiments, objective sensor data 170 may include sensor data associated with the VT headset used in the VR therapy session.

In embodiments, objective sensor data 170 may include data including, but of course not limited to, VR headset tracking, orientation, and/or location data, eye gaze data, headset movement data, headset camera data, headset LIDAR data, headset depth sensor data, and/or any other data generated by sensors, components, external tower sensor data, controller data (e.g., hand or feet-mounted controller data, etc.), and/or software modules of the VR headset.

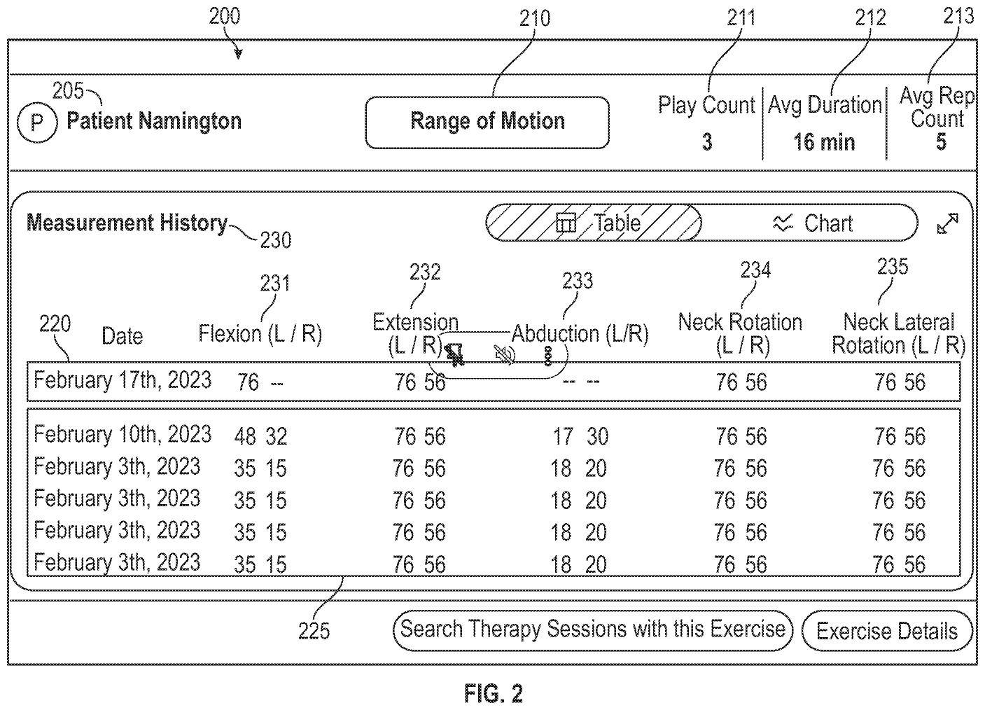

In embodiments, objective sensor data 170 may include data associated with various VR games played by the therapy patient. For example, objective sensor data 170 may include highest level achieved in a particular game, performance statistics related to various VR games (e.g., how fast a level is completed, how many points scored, etc.), level of physical movement during a game, reaction times, range of motion during games, flexion measurements, extension measurements, abduction measurements, neck rotation measurements, neck lateral rotation measurements, etc. In embodiments, different games may generate different data and/or different types of data. For example, FIG. 2 shows an example of a set of objective sensor data for a particular VR game implemented in accordance with embodiments of the present disclosure.

As shown in FIG. 2, VR objective sensor data 200 may include a set of objective sensor data collected for game 210 (which in this example is a “Range of Motion” VR game). In this example, VR objective sensor data 200 may include objective sensor data such as flexion data 231, extension data 232, abduction data 233, neck rotation data 234, and neck lateral rotation data 235. As noted, these data include objective measurement data, rather than subjective impressions. In this case, the collected data may represent data collected during playing of game 210 by player 205. As shown, VR objective sensor data 200 may include data collected during VR therapy session 220, which in this example took place on Feb. 17, 2023. As can be seen, VR objective sensor data 200 may also include an indication of the number of play counts 211 (e.g., the number of times player 205 played game 210 during the therapy session), an average duration 212 (e.g., indicating the average duration of game 210), and an average rep count 213 (e.g., an indication of the average number of reps performed by patient 205 during a play of game 210).

In embodiments, objective sensor data 170 may include historical objective sensor data. For example, objective sensor data 170 may include objective sensor data collected during previous therapy sessions of the therapy patient. For example, as shown in FIG. 2, VR objective sensor data 200 may include historical VR objective sensor data 225, which may include objective sensor data collected during therapy sessions of patient 205 occurring in previously dated therapy sessions. In embodiments, data collector 121 may obtain the historical objective sensor data from an EHR system that may be external to system 100, and/or from database 114.

In some embodiments, VR objective sensor data 200 may be accessible via a graphical user interface and may be presented (e.g., in the format shown in FIG. 2) to a user (e.g., the therapist), such as by output manager 123. For example, VR objective sensor data 200 may be presented as measurement history 230 for game 210 played by patient 205 during several therapy sessions. It is noted that by presenting VR objective sensor data 200 for game 210 for patient 205 as shown in FIG. 2, a therapist can track performance improvements of patient 205 with respect to game 210, as the therapist can glean whether the various measurements show any improvements or regressions over time.

With reference back to FIG. 1, the therapy session data that may be used to generate the therapy notes may include subjective data 171. In embodiments, subjective data 171 may include subjective data that is based on a subjective impression, such as of the therapist and/or the therapy patient, rather than hard objective data generated or measured by one or more sensors (or based on an objective analytical model applied to the sensor data). In this manner, and distinct from objective sensor data 170, subjective data 171 may represent an opinion, impression, and/or belief regarding some aspect of the therapy session that may either not be based on hard measurements or may be based on a subjective interpretation of available data (e.g., sensor data, and/or historical data). For example, subjective data 171 may include subjective observations of the performance or level at which the therapy patient may be performing or has performed an exercise.

In some embodiments, subjective data 171 may include subjective data observed by the therapist that is related to objective data. For example, the therapist may observe a particular aspect of the therapy session and may make, annotate, or otherwise generate a subjective impression related to the particular aspect based on the therapist observation. In this example, objective sensor data may also be generated related to the same particular aspect. For example, objective sensor data including a data point indicating an amount of extension that the therapy patient is able to achieve may be generated for a therapy session. This objective data point may represent an extension level measurement. The therapist may observe the same extension, but may instead characterize the extension in terms of “high,” “low,” “better,” or “worse.” In this case, the same aspect may be associated with an objective data measurement and a subjective impression.

In embodiments, data collector 121 may be configured to enable the therapist to provide at least a portion of subjective data 171 in near-real time during a therapy session. For example, in embodiments, data collector 121 (e.g., in cooperative operation with output manager 123) may provide an interface (e.g., via user terminal 190) via which the therapist may input one or more observations, notes, or impressions related to the therapy session. For example, the therapist may observe a particular event, such as the therapy patient being able to extend a limb to a point that appears longer than before. In embodiments, the interface provided by data collector 121 may enable the therapist to make an input noting the observation. In embodiments, the therapist input may be provided in short format, to minimize the interruption of the therapy session. In some embodiments, the therapist may provide the input via a textual input, an audio input, a selectable menu of potential events, in any available format, and/or in any available way. By providing a mechanism to allow the therapist to make observations in near-real time during a therapy session, system 100 is enhanced tremendously in its ability to tailor or customized the generated therapy notes to the therapist observations.

In some embodiments, data collector 121 may be configured to prompt the therapist to include subjective data. For example, data collector 121 may be configured to prompt questions or instructions to the therapist related particular aspects so that the therapist may provide subjective observations. In some embodiments, the prompts may be in response to a particular event being detected in objective sensor data 170. For example, objective sensor data 170 may indicate a particular issue with an aspect of the therapy session and in this case, data collector 121 may be configured to prompt the therapist for more information, albeit subjective information, related to the particular issue detected.

In some embodiments, in response to an observation by the therapist during the therapy session, data collector 121 may cause objective sensor data to be collected that is related to the observation by the therapist. For example, the therapist may enter an observation that the therapy patient seems to be able to extend a limb to a point that appears longer than before. In response to receiving the therapist observation, data collector may cause one or more sensors to measure and/or record an extension of the limb using one or more sensors available to measure the extension of the limb. In this manner, data collector 121 may collect data that may be used to validate, correct, and/or conform the therapist observation. For example, the collected sensor data may reveal that the patient indeed has improved the extension of the limb, or may revel that the patient has not improved the extension of the limb, in which case the therapy notes generated can be more accurate by providing the confirmed or corrected observations.

In some embodiments, data collector 121 may be configured to enable the therapist to focus the data collection to particular aspects. For example, in embodiments, the therapist may be provided (e.g., via a GUI provided by output manager 123) with a mechanism to indicate particular aspects of the therapy plan for the patient (e.g., therapy goals, expected improvement, etc.). The particular aspects indicated by the therapist may be used by data collector 121 to filter the therapy session data collected. For example, some therapy session data may be irrelevant for a particular therapy goal, in which case data collector 121 may exclude the irrelevant therapy session data from the therapy session data to be used to generate the therapy notes. In a specific example, the therapy goals for a particular patient (e.g., the therapy plan goals and/or the therapy session goals) may include exercises related to improving the therapy patient's range of visual perception. In this example, therapy session data related to arm extension range may not be relevant to the therapy goals, in which case data collector 121 may exclude the irrelevant therapy session data related to arm extension range from the therapy session data to be used to generate the therapy notes. However, data collector 121 may include therapy session data related to the range of visual perception of the therapy patient in the therapy session data to be used to generate the therapy notes.

Therapy notes generator 122 may be configured to generate, based on the therapy session data collected by data collector 121 (e.g., including objective sensor data 170 and/or subjective data 171), the therapy notes. In embodiments, therapy notes generator 122 may be configured to generate the therapy notes by applying the therapy session data collected by data collector 121 to AI model 120.

In embodiments, model 120 may include or represent one or more AI-based models configured to generate therapy notes when applied to content (e.g., therapy session data). Model 120 may represent any model, or any type of model that is configured to generate therapy notes (e.g., based on a particular format) based on a language-based input. For example, a language model may be configured to intake therapy session data and to generate therapy notes in a particular format based on the therapy session data.

In one particular example, therapy notes generator 122 may apply the therapy session data collected by data collector 121 (e.g., including objective sensor data 170 and/or subjective data 171) to model 120 to generate notes in a particular format. In this example, model 120 may be trained to generate therapy notes based on the therapy session data. In embodiments, model 120 may be previously trained based on gold data. In this manner, model 120 may be fully trained to perform operations according to its configuration.

In embodiments, therapy notes generator 122 may enable a user (e.g., the therapist) to customize or configured one or more aspects of the therapy notes generation. For example, therapy notes generator 122 may provide a mechanism (e.g., a GUI provided by output manager 123) to specify configuration parameters, preferences, styles, language, length of the therapy notes, etc. that may be used to format the generated therapy notes. In embodiments, the provided configuration and/or preferences may be used to train model 120 to ensure that the therapy notes generated by model 120 are consistent with the provided configuration and/or preferences. In additional or alternative embodiments, model 120 may learn the user preferences or preferred configuration based on user feedback (e.g., therapy notes corrections provided by the user or explicit feedback by the user) and may generate the therapy notes consistent with the learnt configuration and/or preferences.

In particular embodiments, model 120 may be trained to generate therapy notes in a subjective, objective, assessment, plan (SOAP) format. In the SOAP format, therapy notes may include one or more of at least three sections. A first section may include an objective section configured to include and/or detail an objective assessment including objective performance gains or losses of the therapy patient during the current therapy session (e.g., the therapy session for which the therapy notes are being generated) based on objective sensors data 170. In embodiments, the determination as to whether the therapy patient has experienced a loss or gain, and the level of gain or loss, may be based on current objective sensor data (e.g., objective sensor data collected and/or generated during the current therapy session) and historical objective sensor data (e.g., objective sensor data collected and/or generated during one or more previous therapy sessions). For example, in some embodiments, model 120 may be configured to compare the current objective sensor data to the historical objective sensor data to determine whether a gain or loss in performance of the patient has occurred, and/or the level of gain or loss in performance. In embodiments, model 120 may be configured to translate the objective sensor data 170, which in some embodiments represents raw measurement data, into objective gains or losses of performance into a language expression (e.g., into notes). In this manner, model 120 may be configured to generate the objective section of the therapy notes based on the objective sensor data (e.g., objective sensor data 170).

In a specific example, during a therapy session, a patient may have an increase range of motion for the upper extremity over previous therapy sessions. In this example, objective sensor data 170 may include objective measurements showing the improvement. In this case, the objective section of the therapy notes generated by model 120 may include a linguistic expression explaining that the patient is showing an increase in range of motion for upper extremity. In another example, the amount of time that a therapy patient has spent resting between exercises session over session has decreased, and this may be shown in the objective sensor data 170, which may include historical objective sensor data. In this case, the objective section of the therapy notes generated by model 120 may include a linguistic expression explaining that the therapy patient requires less rest time in-between exercises during the therapy session, which shows an improvement session over session as the therapy patient is able to spend more time performing the therapy exercises rather than resting.

In embodiments, the determination as to whether the therapy patient has experienced a loss or gain, and the level of gain or loss, may be related to the overall therapy session or may be related to a particular exercise (e.g., a particular game or application). For example, first objective sensor data in objective sensor data 170 for a first exercise may show that the patient has improved with respect to the first exercise, but objective sensor data in objective sensor data 170 for all other exercises (or one or more other exercises that are weight more heavily than the first exercise) in the therapy session may show that the patient has a performance loss. In this case, model 120 may determine to generate the objective section of the therapy notes to include an explanation that the therapy patient has experienced a loss in the therapy session. In some other embodiments, model 120 may generate the objective section of the therapy notes to include an explanation that the therapy patient has experienced a gain in one exercise (and may note the exercise) and a loss in other exercises.

A second section of the SOAP format may include an assessment section configured to include and/or detail an assessment of the therapy session based on both objective sensor data 170 and subjective data 171. In embodiments, model 120 may be configured to generate the assessment section of the therapy notes to include notes assessing the therapy session from a perspective of the objective sensor data collected by data collector 121 and the subjective data provided by the therapist (and collected by data collector 121). This objective/subjective assessment may include a more detail picture of the therapy session as may be based not only on the subjective observations or impressions of the therapists, but also on the objective sensor data generated from the one or more sensors of system 100.

A third section of the SOAP format may include a plan section configured to include and/or detail a therapy plan based on, or modified based on, the objective assessment and/or the objective/subjective assessment. For example, an overall therapy plan may be devised for the therapy patient at the start of the therapy. The initial overall therapy plan may include at least a therapy plan for the first therapy session. As the therapy progresses, the overall therapy plan may be modified or updated based on how the therapy is progressing. In embodiments, model 120 may be configured to generate the plan section of the therapy notes to include modifications, if any, to the current overall therapy plan, and/or may include therapy session plans for the next therapy session.

In some embodiments, the plan section may include objective goals (e.g., therapy session goals for next section may include objective gains goals, such as increased number of reps for a particular exercise, increased performance by a certain amount, etc.) and/or subjective goals (e.g., therapy session goals for next section may include ensuring the therapy patient is happy, feels more relaxed and less nervous, etc.). In another example, the plan section may detail that, since the patient is making greater than expected progress in one area of the therapy, a next therapy session may focus on a different area of the therapy.

In embodiments, the therapy notes generated by therapy notes generator 122 may be associated with the therapy session overall, or may be associated with a particular exercise. For example, the therapy notes may provide details related to the overall therapy session, which may include more than one exercise. In this case, the therapy session may be based on therapy data related to all the exercises performed by the therapy patient during the therapy session. In some embodiments, the therapy notes may provide details related to one of the exercises of the therapy session. In this case, the therapy session may be based on therapy data related to the one exercise as performed by the therapy patient during the therapy session.

In embodiments, model 120 may be trained to generate therapy notes that are compliant with not only government requirements, but also insurance requirement or other requirements. For example, in some embodiments, model 120 may be trained to generate therapy notes that are compliant with auditing requirements.

It is noted that, although the process for training model 120 in accordance with embodiments herein has not been described in extremely deep technical details for the sake of brevity, and because techniques for training AI models is not within the scope of this disclosure, those of skilled in the art will recognize that training an AI model may entail training the AI model using gold data that has been tailored to ensure that the AI model is trained to generate an output in accordance with the gold data used. As such, it is noted that the present disclosure complies with all enablement and/or description requirements.

Output manager 123 may be configured to manage the outputting of the therapy notes generated by therapy generator 122. In embodiments, output manager 123 may be configured to provide the therapy notes to at least one entity according to configuration (e.g., user or system configuration stored in database 114). For example, in some embodiments, the therapy notes may be provided to an EHR system that is external to system 100, or may be provided to an insurance agency. In this manner, system 100 may be integrated into existing EHR systems. In some embodiments, output manager 123 may be configured to provide the therapy notes in an alert (e.g., text message, email, etc.) provided to an entity based on the configuration. In embodiment, output manager 123 may be configured to provide the therapy notes to a user (e.g., the therapist, a doctor, etc.) using user terminal 190, such as via a presentation to the user (e.g., via a GUI displayed on user terminal 190).

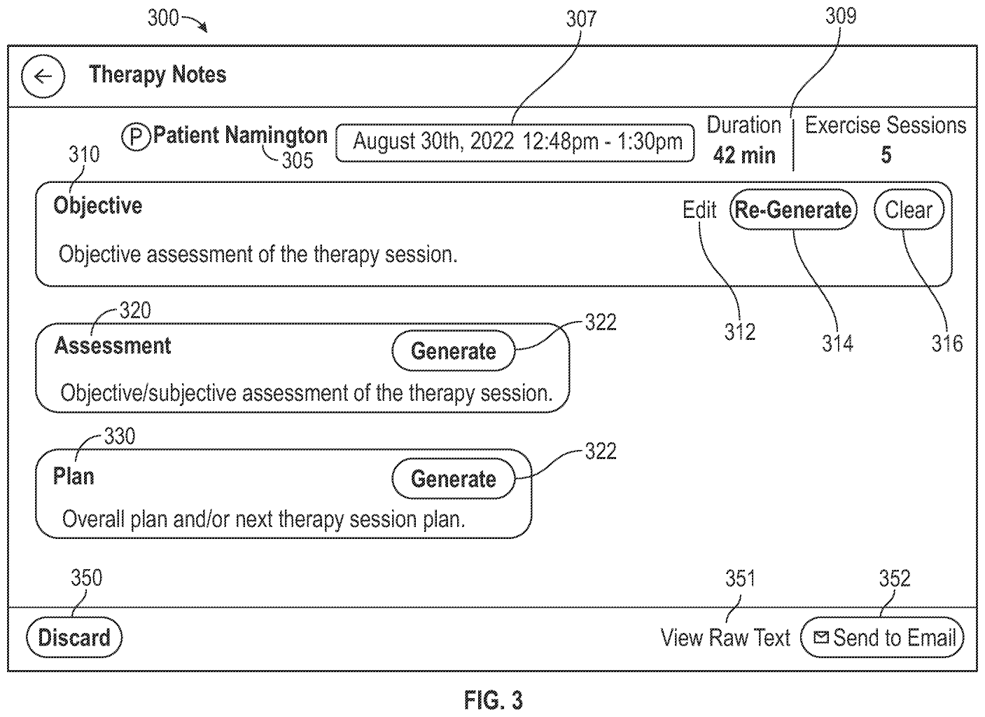

FIG. 3 shows an example a GUI 300 configured with capabilities and functionality for presenting therapy notes in accordance with embodiments of the present disclosure. As shown in FIG. 3, GUI 300 may be configured to present therapy notes in the SOAP format in accordance with embodiments of the present disclosure. In particular, GUI 300 may include element 305 for displaying the name of the therapy patient, element 307 for displaying the date of the therapy session for which the therapy notes are generated. GUI 300 may include section 309 configured for displaying details of the therapy session, such as duration of the therapy session and/or number of exercises performed by the patient during the therapy session.

In embodiments, GUI 300 may include objective section 310 configured to include and/or detail an objective assessment including objective performance gains or losses of the therapy patient during the current therapy session (e.g., the therapy session for which the therapy notes are being generated) based on objective sensors data 170 in accordance with embodiments of the present disclosure, assessment section 320 configured to include and/or detail an assessment of the therapy session based on both objective sensor data 170 and subjective data 171, and plan section 330 configured to include and/or detail a therapy plan based on, or modified based on, the objective assessment and/or the objective/subjective assessment.

In some embodiments, objective section 310, assessment section 320, and plan section 330 may be generated concurrently upon request by a user (e.g., via an element not shown of GUI 300). In other embodiments, each of objective section 310, assessment section 320, and plan section 330 may be configured to be generated individually. For example, an interactive element may be provided for each of objective section 310, assessment section 320, and plan section 330 for requesting that the respective section be generated. For example, assessment section 320 may include element 322 configured for requesting that assessment section 320 be generated. In embodiments, upon a user selecting element 322, system 100 may operate to generate the assessment section of the therapy section for the selected therapy section in accordance with the description herein. The generated assessment section may be displayed in assessment section 320 of GUI 300. In this same example, plan section 330 may include element 332 configured for requesting that plan section 330 be generated. In embodiments, upon a user selecting element 332, system 100 may operate to generate the plan section of the therapy section for the selected therapy section in accordance with the description herein. The generated plan section may be displayed in plan section 330 of GUI 300.

In embodiments, GUI 300 may be configured to allow modification of each section of the therapy notes. For example, in the example shown in FIG. 3, objective section 310 may already be generated and displayed. In this case, element 312 may be provided allowing a user to edit the content in the objective session of the therapy notes. In embodiments, editing the objective section of the therapy notes may include changing the language or phrases used in the objective section, making corrections to the contents of the objective section, replacing words, changing the format of the contents of the objective section, etc. Element 316 may be configured to clear objective section 310 to remove the contents displayed therein.

In embodiments, element 314 may enable the user to re-generate the content in objective section 310 of the therapy notes. For example, a user may activate element 314, in which case the therapy session data collected by data collector 121, which may include objective sensor data 170 and subjective data 171, may be resubmitted to model 120 to generate the therapy notes again. In some embodiments, the regeneration may regenerate only the objective section 310, but may leave any other already generated sections alone. The regenerated content may be displayed in objective section 310.

In embodiments, output manager 123 may provide a feedback mechanism to enable model 120 to refine its learning. For example, the user may edit a section of the therapy notes and may make changes to the content of the section. The changes to the content may be used to train model 120 based on the changes to, next time, generate content that takes into account the changes made. In some embodiments, for example where regeneration of a section is requested, the regeneration may be used to further train model 120. In embodiments, upon a re-generation request, output manager 123 may prompt the user to provide information on a reason for the re-generation request, including specific portions of the content that prompted the user to request the re-generation. The user feedback may be provided to model 120 to further train model 120. In this manner, model 120 may learn and may generate content that is more consistent with user preferences.

GIU 300 may also include element 350 for discarding the therapy notes, element 351 for requesting and displaying the raw text of the therapy notes rather than the SOAP formatted therapy notes, and element 352 to cause the therapy notes to be provided (e.g., via email) to one or more entities.

With reference back to FIG. 1, user terminal 190 may include a mobile device, a smartphone, a tablet computing device, a personal computing device, a laptop computing device, a desktop computing device, a computer system of a vehicle, a personal digital assistant (PDA), a smart watch, another type of wired and/or wireless computing device, or any part thereof. In embodiments, user terminal 190 may be configured to provide a user interface (e.g., a graphical user interface (GUI)) structured to facilitate an operator interacting with system 100, e.g., via network 145, to execute and leverage the features provided by server 110. In embodiments, the operator may be enabled, e.g., through the functionality of user terminal 190, to provide configuration parameters that may be used by system 100 to provide functionality for generating therapy notes in accordance with embodiments of the present disclosure, and/or to view and/or interact with the generated therapy notes in various formats and based on various functionalities (e.g., as described with respect to output manger 123 and FIG. 3). In embodiments, user terminal 190 may be configured to communicate with other components of system 100.

In embodiments, network 145 may facilitate communications between the various components of system 100 (e.g., server 110 and/or user terminal 130). Network 145 may include a wired network, a wireless communication network, a cellular network, a cable transmission system, a Local Area Network (LAN), a Wireless LAN (WLAN), a Metropolitan Area Network (MAN), a Wide Area Network (WAN), the Internet, the Public Switched Telephone Network (PSTN), etc.

In general terms, embodiments of the present disclosure provide functionality for automatically generating therapy notes for a therapy session of a therapy patient that go beyond capabilities of current systems. As has been noted above, current systems are not able to address many deficiencies related to the current process for generating therapy notes for therapy sessions. In particular, and quite importantly, current systems do not collect and are not able to integrate objective sensor data into the therapy notes generation process. Embodiments of the present disclosure allow for such an integration, which greatly enhances the therapy notes generation process.

One particular application of the techniques and systems disclosed herein may be in a VR therapy environment. As noted above, a VR therapy session may include the use of a VR headset to immerse the therapy patient in a virtual environment. Embodiments of the present disclosure may be applicable in a VR therapy environment, in which the exercises of a therapy session may include different VR apps or games. For example, a first VR game or app may be used by the therapy patient to perform a first exercise. Therefore, within the context of the therapy notes generation process of embodiments for VR therapy sessions, an exercise may refer to a VR game or app, and the objective sensor data may include not only objective therapy session data, but also VR headset related data, VR game related data, etc.

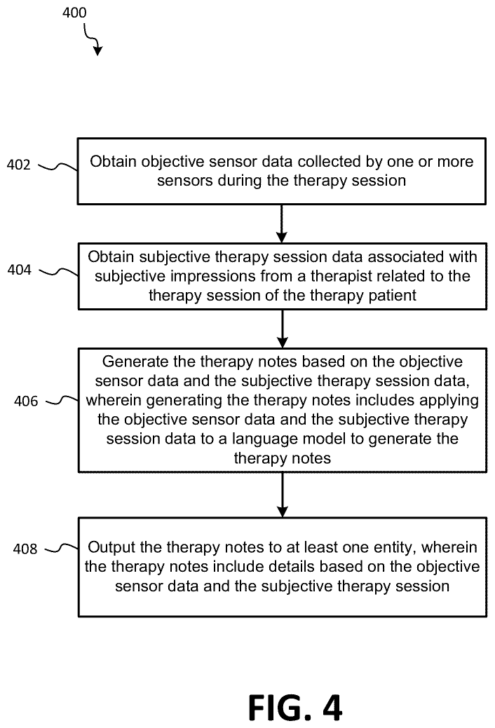

FIG. 4 shows a high-level flow diagram 400 of operation of a system configured for providing functionality for automatically generating therapy notes for a therapy session of a therapy patient in accordance with embodiments of the present disclosure. For example, the functions illustrated in the example blocks shown in FIG. 4 may be performed by system 100 of FIG. 1 according to embodiments herein. In embodiments, the operations of the method 400 may be stored as instructions that, when executed by one or more processors, cause the one or more processors to perform the operations of the method 400.

At block 402, objective sensor data collected by one or more sensors during the therapy session may be obtained. In embodiments, functionality of a data collector (e.g., data collector 121 as shown in FIG. 1) may be used to obtain the objective sensor data (e.g., objective sensor data 170 as shown in FIGS. 1 and 2) collected by one or more sensors during the therapy session according to operations and functionality as described above with reference to data collector 121 and as illustrated in FIG. 1.

At block 404, subjective therapy session data associated with subjective impressions from a therapist related to the therapy session of the therapy patient may be obtained. In embodiments, functionality of a data collector (e.g., data collector 121 as shown in FIG. 1) may be used to obtain the subjective therapy session data (e.g., subjective data 171 as shown in FIG. 1) associated with subjective impressions from a therapist related to the therapy session of the therapy patient according to operations and functionality as described above with reference to data collector 121 and as illustrated in FIG. 1.

At block 406, the therapy notes are generated based on the objective sensor data and the subjective therapy session data. In embodiments, generating the therapy notes may include applying the objective sensor data and the subjective therapy session data to a language model to generate the therapy notes. In embodiments, functionality of a therapy notes generator (e.g., therapy notes generator 122 as shown in FIG. 1) may be used to generate the therapy notes based on the objective sensor data and the subjective therapy session data according to operations and functionality as described above with reference to therapy notes generator 122 and as illustrated in FIG. 1.

At block 408, the therapy notes are output to at least one entity. In embodiments, the therapy notes may include details based on the objective sensor data and the subjective therapy session. In embodiments, functionality of an output manager (e.g., output manager 123 as shown in FIG. 1) may be used to output the therapy notes to at least one entity according to operations and functionality as described above with reference to output manager 123 and as illustrated in FIG. 1.

Persons skilled in the art will readily understand that advantages and objectives described above would not be possible without the particular combination of computer hardware and other structural components and mechanisms assembled in this inventive system and described herein. Additionally, the algorithms, methods, and processes disclosed herein improve and transform any general-purpose computer or processor disclosed in this specification and drawings into a special purpose computer programmed to perform the disclosed algorithms, methods, and processes to achieve the aforementioned functionality, advantages, and objectives. It will be further understood that a variety of programming tools, known to persons skilled in the art, are available for generating and implementing the features and operations described in the foregoing. Moreover, the particular choice of programming tool(s) may be governed by the specific objectives and constraints placed on the implementation selected for realizing the concepts set forth herein and in the appended claims.

The description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the inventions can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification

Those of skill in the art would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various embodiments of the present disclosure may be combined or performed in ways other than those illustrated and described herein.

Functional blocks and modules in FIGS. 1-4 may comprise processors, electronics devices, hardware devices, electronics components, logical circuits, memories, software codes, firmware codes, etc., or any combination thereof. Consistent with the foregoing, various illustrative logical blocks, modules, and circuits described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the disclosure herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal, base station, a sensor, or any other communication device. In the alternative, the processor and the storage medium may reside as discrete components in a user terminal.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. Computer-readable storage media may be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, a connection may be properly termed a computer-readable medium. For example, if the software is transmitted from a website, server, or other remote source using a coaxial cable, fiber optic cable, twisted pair, or digital subscriber line (DSL), then the coaxial cable, fiber optic cable, twisted pair, or DSL, are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of automatically generating therapy notes for a therapy session of a therapy patient, comprising:
obtaining objective sensor data collected by one or more sensors during the therapy session;
obtaining subjective therapy session data associated with subjective impressions from a therapist related to the therapy session of the therapy patient;
generating the therapy notes based on the objective sensor data and the subjective therapy session data, wherein generating the therapy notes includes applying the objective sensor data and the subjective therapy session data to a language model to generate the therapy notes; and
outputting the therapy notes to at least one entity, wherein the therapy notes include details based on the objective sensor data and the subjective therapy session.

2. The method of claim 1, wherein the therapy session includes at least one therapy exercise performed using a virtual reality (VR) headset configured to present a virtual environment to the patient for performing a virtual action associated with at least one physical action of the at least one therapy exercise.

3. The method of claim 2, wherein the one or more sensors include at least one sensor associated with the VR headset.

4. The method of claim 1, wherein the objective sensor data includes sensor data collected by at least one sensor during at least one previous therapy session of the therapy patient.

5. The method of claim 1, wherein the subjective therapy session data includes subjective data associated with subjective impressions from the therapist related to at least one previous therapy session of the therapy patient.

6. The method of claim 5, wherein the subjective data associated with subjective impressions from the therapist related to the at least one previous therapy session of the therapy patient is extracted from therapy notes associated with the at least one previous therapy session of the therapy patient.

7. The method of claim 1, wherein the subjective therapy session data includes subjective data collected during the therapy session.

8. The method of claim 7, wherein the subjective data collected during the therapy session includes one or more of:
spontaneous observations made by the therapist during the therapy session;
audio notes made by the therapist during the therapy session; and
text notes entered by the therapist during the therapy session.

9. The method of claim 1, wherein the therapy notes include one or more of:
an objective section configured to include an objective indication of improvement of performance of the therapy patient during the therapy session based, at least in part, on the objective sensor data;
an assessment section configured to include an assessment of the therapy session based on both the objective sensor data and the subjective therapy session data; and
a plan section configured to include at least a portion of an overall therapy plan for the therapy patient based on one or more of:
the improvement of performance of the therapy patient during the therapy session; and
the assessment of the therapy session.

10. The method of claim 1, wherein the at least one entity include:
an external electronics health record; and
the therapist.

11. A system for automatically generating therapy notes for a therapy session of a therapy patient, comprising:
at least one processor; and
a memory operably coupled to the at least one processor and storing processor-readable code that, when executed by the at least one processor, is configured to perform operations including:
obtaining objective sensor data collected by one or more sensors during the therapy session;
obtaining subjective therapy session data associated with subjective impressions from a therapist related to the therapy session of the therapy patient;
generating the therapy notes based on the objective sensor data and the subjective therapy session data, wherein generating the therapy notes includes applying the objective sensor data and the subjective therapy session data to a language model to generate the therapy notes; and
outputting the therapy notes to at least one entity, wherein the therapy notes include details based on the objective sensor data and the subjective therapy session.

12. The system of claim 11, wherein the therapy session includes at least one therapy exercise performed using a virtual reality (VR) headset configured to present a virtual environment to the patient for performing a virtual action associated with at least one physical action of the at least one therapy exercise, and wherein the one or more sensors include at least one sensor associated with the VR headset.

13. The system of claim 11, wherein the objective sensor data includes sensor data collected by at least one sensor during at least one previous therapy session of the therapy patient.

14. The system of claim 11, wherein the subjective therapy session data includes subjective data associated with subjective impressions from the therapist related to at least one previous therapy session of the therapy patient.

15. The system of claim 14, wherein the subjective data associated with subjective impressions from the therapist related to the at least one previous therapy session of the therapy patient is extracted from therapy notes associated with the at least one previous therapy session of the therapy patient.

16. The system of claim 11, wherein the subjective therapy session data includes subjective data collected during the therapy session.

17. The system of claim 16, wherein the subjective data collected during the therapy session includes one or more of:
spontaneous observations made by the therapist during the therapy session;
audio notes made by the therapist during the therapy session; and
text notes entered by the therapist during the therapy session.

18. The system of claim 11, wherein the therapy notes include one or more of:
an objective section configured to include an objective indication of improvement of performance of the therapy patient during the therapy session based, at least in part, on the objective sensor data;
an assessment section configured to include an assessment of the therapy session based on both the objective sensor data and the subjective therapy session data; and
a plan section configured to include at least a portion of an overall therapy plan for the therapy patient based on one or more of:
the improvement of performance of the therapy patient during the therapy session; and
the assessment of the therapy session.

19. The system of claim 11, wherein the at least one entity include:
an external electronics health record; and
the therapist.

20. A computer-based tool for automatically generating therapy notes for a therapy session of a therapy patient, the computer-based tool including non-transitory computer readable media having stored thereon computer code which, when executed by a processor, causes a computing device to perform operations comprising:
obtaining objective sensor data collected by one or more sensors during the therapy session;
obtaining subjective therapy session data associated with subjective impressions from a therapist related to the therapy session of the therapy patient;
generating the therapy notes based on the objective sensor data and the subjective therapy session data, wherein generating the therapy notes includes applying the objective sensor data and the subjective therapy session data to a language model to generate the therapy notes; and
outputting the therapy notes to at least one entity, wherein the therapy notes include details based on the objective sensor data and the subjective therapy session.
